(19) United States
(12) Patent Application Publication
(10) Pub. No.: US 2004/0091937 A1
(43) Pub. Date: May 13, 2004

(54) Use of fluorine NMR for high throughput screening

(76) Inventors: Claudio Dalvit, Milan (IT); Brian J. Stockman, Waterford, CT (US); Maria Flocco, Milan (IT); Marina Veronesi, Milan (IT)

Correspondence Address: MUETING, RAASCH & GEBHARDT, P.A., P.O. BOX 581415, MINNEAPOLIS, MN, 55458, US

(21) Appl. No.: 10/455,077

(22) Filed: Jun. 5, 2003

Related U.S. Application Data
(60) Provisional application No. 60/454,765, filed on Mar. 14, 2003.
(60) Provisional application No. 60/454,766, filed on Mar. 14, 2003.
(60) Provisional application No. 60/398,875, filed on Jul. 26, 2002.
(60) Provisional application No. 60/389,252, filed on Jun. 17, 2002.
(60) Provisional application No. 60/386,897, filed on Jun. 5, 2002.

(51) Int. Cl. G01N 33/53

(52) U.S. Cl. 435/7.1

(57) ABSTRACT

High-Throughput ligand-based NMR screening with competition binding experiments using ¹⁹F detection.

This application claims the benefit of U.S. Provisional Application Serial No. 60/454,766, filed 14 Mar. 2003 which is incorporated herein by reference in its entirety.

### BACKGROUND OF THE INVENTION

Many drugs currently on the market were developed from leads identified from high throughput screening (HTS). Targets of therapeutic interest used in HTS are often recombinant proteins produced from cloned genes which can be expressed in different ways. A large compound collection is typically screened against these proteins for the identification of inhibitors.

During the last ten years the size of the proprietary compound collection has increased exponentially as a result of systematic application of combinatorial chemistry to different projects. Combinatorial chemistry nowadays generates large compound libraries that complement other compound libraries available from traditional medicinal chemistry and natural sources. The development and application of robotics and automation have made it feasible to test large numbers of compounds in a short period of time. Several new detection systems are used for the identification of potential lead molecules.

Recently, nuclear magnetic resonance (NMR) has emerged as a powerful method for the detection of small molecules that interact with targets of pharmaceutical interest. Although NMR is not a sensitive technique, it has the advantage that it is less subject to artifacts observed with other systems of detection. Recent developments in cryogenic NMR probe technology have reduced the period of time or the amount of protein necessary for the screening.

NMR methods have been used for screening a large compound collection against isotopically labeled proteins. Chemical shift changes of cross peaks in a ¹⁵N—¹H HSQC spectrum of the target protein are monitored in the presence of a compound mixture. Deconvolution of the mixture then results in the identification of the molecule interacting with the protein (i.e., the compound responsible for the chemical shift changes). When the three dimensional structure of the protein is known and the sequence specific NMR assignments of the protein backbone resonances have been obtained, the method provides important structural information of the ligand binding site and ligand binding mode.

Another method for performing NMR screening is based on the detection of the ligand resonances. Several NMR parameters have been proposed in the literature as a tool for ligand identification. These methodologies permit rapid deconvolution of the screened mixtures and are particularly suited for the identification of medium to low affinity ligands.

However, these techniques suffer from some drawbacks. First, no structural information regarding the binding site is directly available. Second, high affinity ligands and molecules that bind covalently to the receptor escape detection because of the large excess of the test compound over protein typically used in these experiments. That is, compounds interacting tighter to the protein or compounds that have a slow kinetics will not be detected because the residence time of these compounds within the protein is longer than the window of the mixing time (e.g., 1 to 2 seconds) employed in the NMR experiments. Third, compounds with poor solubilities that are potential ligands are difficult to detect since the method requires the observation of the ligand signals.

Thus, what is needed are additional NMR methods that can be used to detect ligands to target molecules, such as proteins, without the drawbacks associated with typical ligand-observed screening experiments.

### SUMMARY OF THE INVENTION

The present invention is related to rational drug design. Specifically, the present invention provides a nuclear magnetic resonance (NMR) method of screening for compounds that interact with a target molecule (e.g., typically a protein). The method involves the use of ¹⁹F NMR, particularly ¹⁹F NMR competition binding experiments, to detect the binding interaction.

Competition binding experiments involve the displacement of a reference compound in the presence of a competitive molecule. Preferably, the reference compound binds to the target molecule with a binding affinity in the micromolar range. Preferably, the test compound interacts with the target molecule with a binding affinity stronger than 1 micromolar (e.g., in the nanomolar range), although compounds binding with binding affinities of weaker than (i.e., more than) 1 micromolar can also be evaluated using the methods of the present invention.

The present methodology, particularly when it involves competition binding experiments, can be used for performing efficient high throughput screening (HTS) based on properly set-up competition binding experiments without the drawbacks associated with typical ligand-observed screening experiments. In addition, the methods provide an estimation of the K_{D} of the identified ligand using a single point measurement. With this approach it is possible to screen thousands of compounds in a short period of time against protein or DNA and RNA fragments, for example.

The present invention could also find useful applications for rapid screening of chemical mixtures (i.e., mixtures of two or more test compounds) such as plant and fungi extracts. Rapid screening techniques typically involve providing a plurality of test samples, each test sample comprising a mixture of two or more test compounds.

Methods of the present invention involve identifying a ligand to a target molecule using at least the following steps: providing an ¹⁹F-labelled reference compound that interacts with the target molecule; collecting a 1D ¹⁹F nuclear magnetic resonance spectrum of the ¹⁹F-labelled reference compound in the presence of the target molecule; providing a test sample (preferably a plurality of test samples) comprising at least one test compound; collecting a 1D ¹⁹F nuclear magnetic resonance spectrum of the ¹⁹F-labelled reference compound in the presence of each test sample and the target molecule; comparing the spectrum of the ¹⁹F-labelled reference compound in the presence of the target molecule to the spectrum of the ¹⁹F-labelled reference compound in the presence of each test sample and the target molecule to determine a change in one or more of the ¹⁹F-labelled reference compound resonances; and identifying at least one test compound that interacts with the target molecule, wherein the test compound displaces the ¹⁹F-labelled reference compound. A test compound (i.e., a potential ligand) is a ligand if it displaces the reference compound from the target molecule.

Preferably, methods of the present invention include a step of identifying the reference compound comprising: collecting a WaterLOGSY nuclear magnetic resonance spectrum of a potential reference compound in the absence of the target molecule; collecting a WaterLOGSY nuclear magnetic resonance spectrum of the potential reference compound in the presence of the target molecule; and comparing the WaterLOGSY spectra to identify whether the potential reference compound interacts with the target molecule.

For certain embodiments of the methods of the present invention, the reference compound is provided in combination with an ERETIC signal with defined linewidth, amplitude, and frequency. For these methods, collecting a ID ¹⁹F nuclear magnetic resonance spectrum of the ¹⁹F-labelled reference compound in the presence of the target molecule includes collecting a spectrum of the ¹⁹F-labelled reference compound with the ERETIC signal in the presence of the target molecule; and collecting a 1D 19F nuclear magnetic resonance spectrum of the ¹⁹F-labelled reference compound in the presence of each test sample and the target molecule includes collecting a spectrum of the ¹⁹F-labelled reference compound with the ERETIC signal in the presence of each test sample and the target molecule.

For certain embodiments of the methods of the present invention, the ¹⁹F-labelled reference compound is provided in combination with an ¹⁹F-labelled non-interacting compound. For these methods, collecting a 1D ¹⁹F nuclear magnetic resonance spectrum of the ¹⁹F-labelled reference compound in the presence of the target molecule includes collecting a spectrum of the ¹⁹F-labelled reference compound and the ¹⁹F-labelled non-interacting compound in the presence of the target molecule; and collecting a 1D ¹⁹F nuclear magnetic resonance spectrum of the ¹⁹F-labelled reference compound in the presence of each test sample and the target molecule includes collecting a spectrum of the ¹⁹F-labelled reference compound and the ¹⁹F-labelled non-interacting compound in the presence of each test sample and the target molecule.

In another embodiment, the present invention provides a method of screening compounds to identify a ligand to a target molecule. The method includes: collecting a first 1D ¹⁹F nuclear magnetic resonance spectrum of at least one test compound; exposing the at least one test compound to a target molecule; collecting a second 1D ¹⁹F nuclear magnetic resonance spectrum of the at least one test compound that has been exposed to the target molecule; and comparing the first and second spectra to determine a change in one or more of the resonances and identify at least one test compound that interacts with the target molecule.

### BRIEF DESCRIPTION OF THE DRAWINGS

FIG. 1. Difference in linewidth due to CSA interaction of the ¹⁹F signal of a small molecule free in solution and when bound to a large macromolecule as a function of the ¹⁹F Larmor frequency. This simulation was performed using the last term of equation 1 in the assumption of an axally symmetric CSA tensor with a ¹⁹F CSA of 100 ppm and a correlation time τ_{c} of 200 ps for the small molecule when free in solution. Different correlation times for the macromolecule corresponding to different sizes of the macromolecule were considered (values indicated with the curves) The dashed vertical lines indicate some of the commercially available spectrometers. The value corresponding to the ¹H Larmor frequency of these spectrometers are indicated with the vertical lines.

FIG. 2. NMR screening and deconvolution performed with 50 μM of the weak affinity ligand Compound A (K_{D}=10 μM) of the p21 activated kinase. The reference molecule contains a CF₃ group bound to a six member aromatic ring. The chemical shifts are referenced to TFA. The two spectra were recorded without (left) and with a spin-echo scheme with τ=0.1 s (right). The spectra were acquired in the presence of 1.5 μM of the protein for the spy molecule alone (a), in the presence of a 20 μM seven compound mixture containing the molecules SPECS AB-323/25048456 (supplied by SPECS, Rijswijk, the Netherlands) ethyl 2-quinoxalinecarboxylate, methyl isoquinoline-3-carboxylate, 7-phenyl-4-pteridinol, 2-amino-6-methylquinazolin-4-ol, 5-methylbenzimidazole and Compound B (b), in the presence of the chemical mixture without Compound B (c), in the presence of only Compound B (d). The spectrum of the reference compound in PBS in the absence of the protein is shown in (e). A total of 128 scans with a repetition time of 3.1 s were acquired for each experiment.

FIG. 3. One dimensional ¹⁹F spectra recorded in the presence of the weak-affinity ligand Compound A for the p21 activated kinase and the non-interacting trifluoroacetic acid (TFA) molecule. The chemical shifts are referenced to TFA. A total of 128 scans with a repetition time of 3.1 s were acquired for each experiment. The concentration of Compound A and TFA were 50 and 15 μM, respectively. The spectra were recorded in the absence (a) and the presence of 1.5 μM of the protein (b). The spectrum in (c) corresponds to the difference of the two spectra in (a) and (b). The only signal present in the difference spectrum originates from the spy molecule.

FIG. 4. HTS and deconvolution performed with one Dimensional ¹⁹F spectra recorded in the presence of the weak-affinity ligand Compound A for the p21 activated kinase and the non-interacting trifluoroacetic acid (TFA) molecule. The chemical shifts are referenced to TFA. (a-d) NMR screening and deconvolution performed with 50 μM of Compound A and 15 μM of TFA. (a) Spectrum recorded in the absence of the protein, (b-d) spectra recorded in the presence of 1.5 μM of the protein. (b) Spectrum recorded in the absence of the mixture, (c) spectrum recorded in the presence of a 20 μM six compound mixture containing the molecules SPECS AB-323/25048456, ethyl 2-quinoxalinecarboxylate, methyl isoquinoline-3-carboxylate, 7-phenyl-4-pteridinol, 2-amino-6-methylquinazolin-4-ol, 5-methylbenzimidazole, (d) spectrum recorded in the presence of the same chemical mixture with the addition of 20 μM Compound B. The presence of the competing molecule Compound B results in almost complete displacement of the reference compound from the protein (d) and the spectrum is similar to the spectrum for the two molecules in PBS (a).

FIG. 5. Percentage of molecules containing an F atom within the MDDR library. The search was performed from year 1981 to year 2000 in time intervals of five years. The percentage for each interval is indicated above the bars.

FIG. 6. ¹⁹F spin-echo spectra recorded as a function of the HSA concentration. The CF₃ resonance of the control molecule (2) is at +15.46 ppm and the CF₃ resonance of the spy molecule (1) is at +14.62 ppm. The spectra were acquired with a total spin-echo period of 320 ms with an interval between the 1800 pulses (2τ) of 40 ms. A total of 96 scans with a repetition time of 3.5 s and a spectral width of 25 ppm were acquired for each spectrum. The data were multiplied with an exponential function of 1 Hz before Fourier transformation. The concentration of the two molecules was 25 μM whereas the concentration for HSA was from top to bottom, 0, 300, 500, 700 and 900 nM. The signal intensity ratio I(1)/I(2) is from top to bottom, 0.86, 0.66, 0.38, 0.21 and 0.07.

FIG. 7. ¹⁹F spin-echo spectra recorded as a function of the HSA concentration. The CF resonance of the reference molecule (3) is at −64.06 (lower spectra) and the CF₃ resonance of the control molecule (2) is at +15.46 ppm (upper spectra). The spectra were acquired with a total spin-echo period of 80 ms with an interval between the 180° pulses (2τ) of 40 ms. A total of 96 scans were recorded for the lower spectra and 64 scans for the upper spectra with a repetition time of 3.5 s and a spectral width of 25 ppm. The data were multiplied with an exponential function of 1 Hz before Fourier transformation. The concentration of (3) and (2) was 50 and 25 μM, respectively whereas the concentration for HSA was from left to right, 0, 150, 300, 450, 600 nM. The signal intensity ratio I(3)/I(2) at the plotted scale intensity is from left to right, 0.94, 0.69, 0.53, 0.36 and 0.25.

FIG. 8. Plot of the signal intensity ratio (x axis) of the two ¹⁹F signals of FIG. 7 as a function of the fraction of bound reference molecule ([EL]/[L_{TOT}]) (y axis). The last point on the right corresponds to the value in the absence of the protein. Two ratios ([EL]/[L_{TOT}]) were calculated as previously described using the limits of the ITC-derived K_{D} value of 41±3.3 μM for (3). Values indicated by circles were calculated with a K_{D} of 44.3 μM, values indicated by squares were calculated with a K_{D} of 37.7 μM. The curves represent the best fits of the experimental points.

FIG. 9.¹⁹F NMR screening performed with the control molecule (2) (top) and the spy molecule (3) (bottom). The spectra were recorded with a total spin-echo period of 160 ms with an interval between the 180° pulses (2τ) of 40 ms. A total of 96 scans were recorded with a repetition time of 3.5 s and a spectral width of 25 ppm. The data were multiplied with an exponential function of 1 Hz before Fourier transformation. The concentration of (3) and (2) was 50 and 25 μM, respectively. The spectra on the left were recorded in the absence of protein while all the other spectra were recorded in the presence of 600 nM HSA. The latter were recorded in the absence of a chemical mixture (2^{nd} from left), in the presence of 50 μM 5-CH₃ D,L Trp and Sucrose (3^{rd} from left) and in the presence of 50 μM 5-CH₃ D,L Trp, Sucrose and 25 μM of (4) (right).

FIG. 10. Detection limits of ¹⁹F NMR screening. Experiments performed with the control molecule (2) (top) and the spy molecule (3). The spectra were recorded with a total spin-echo period of 320 ms (top) and 1.2 s (bottom) with an interval between the 180° pulses (2τ) of 40 ms. A total of 64 (top) and 128 (bottom) scans were recorded with a repetition time of 3.5 s and a spectral width of 25 ppm. The data were multiplied with an exponential function of 1 Hz before Fourier transformation. The concentration of (3) and (2) was 50 and 25 μM, respectively. The spectra on the left were recorded in the absence of protein while all the other spectra were recorded in the presence of only 150 nM HSA. The latter were recorded in the absence of a chemical mixture (2^{nd} from left), and in the presence of a mixture containing 25 μM of (4) (right).

FIG. 11. ¹⁹F NMR screening performed in the presence of non-deuterated buffers and detergents. (top) Proton spectrum of a 600 nM solution of HSA in 100 mM HEPES and 1% Glycerol and in the presence of 50 μM of the spy molecule (3) and 25 μM of the control molecule (2). After water suppression the only visible signals are those of the buffer and glycerol. A total of 128 scans was recorded with a repetition time of 2.7 s. (Bottom) ¹⁹F spectra recorded for the same solution in the absence (1^{st} and 3^{rd} spectra from left) and in the presence (2^{nd} and 4^{th} spectra from left) of a mixture containing 25 μM (4). The spectra were recorded with a total spin-echo period of 160 ms with an interval between the 180° pulses (2τ) of 40 ms. A total of 64 (left) and 128 (right) scans were recorded with a repetition time of 3.5 s and a spectral width of 25 ppm. The data were multiplied with an exponential function of 1 Hz before Fourier transformation.

FIG. 12. Structures of Compounds 1-4.

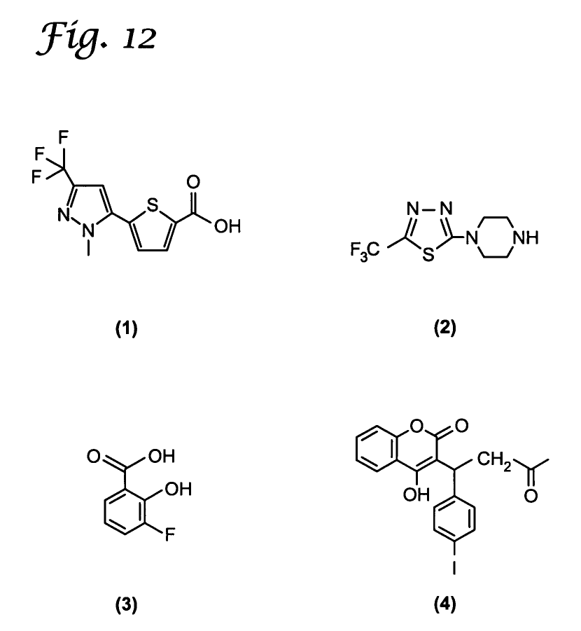

### DETAILED DESCRIPTION OF ILLUSTRATIVE EMBODIMENTS OF THE INVENTION

The present invention is directed to the use of ¹⁹F NMR, particularly ¹⁹F NMR competition binding experiments. That is, the present invention is directed to ligand-based screening (preferably, competition screening) using ¹⁹F experiments. Fluorine-19 detection has many advantages over proton detection in these experiments.

Fluorine is a favorable nucleus for these experiments because of the significant Chemical Shift Anisotropy (CSA) contribution to the ¹⁹F transverse relaxation of the ligand signal when bound to a protein. That is, the CSA contribution to the ¹⁹F transverse relaxation makes the fluorine signal especially responsive to the effects of complex formation with the target. A low to moderate affinity ligand containing an ¹⁹F atom can be used as a reference molecule for the detection and characterization of new ligands. Also, the detection of fluorine significantly reduces or even eliminates the problem of spectral overlap, which occurs in proton (¹H) NMR, as the vast majority of compounds to be tested will not contain a fluorine atom. Like proton NMR, ¹⁹F-NMR is highly sensitive and is amenable to rapid data collection, enabling the high-throughput screening of large compound libraries. Fluorine is often used in drug-design efforts to enhance the pharmacokinetic properties of biologically active compounds. As about 12% of the molecules comprising the Available Chemical Directory Screening Compounds (ACD-SC) contain a fluorine atom, a reference compound for the competition screening can typically be obtained without recourse to chemical synthesis. In fact, the fluoro-benzene and the trifluoromethyl-benzene moiety are found in approximately 150,000 and approximately 40,000 molecules, respectively.

Competition binding experiments involve the displacement of a reference compound in the presence of a competitive molecule. Preferably, the reference compound binds to the target molecule with a binding affinity in the micromolar range. Preferably, the test compound binds to the target molecule with a binding affinity stronger than (i.e., less than) 1 micromolar (e.g., in the nanomolar range), although compounds binding with a binding affinity weaker than (i.e., more than) 1 micromolar can also be evaluated using the methods of the present invention.

Although the methods described herein are particularly useful for identifying ligands that are relatively strong binders to the target molecule, they can be used for identifying ligands of a wide range of binding affinities. The relatively strong binders are typically defined as those having a dissociation binding constant K_{D} of less than about 1 micromolar, preferably less than about 500 nM, more preferably less than about 100 nM.

Competition binding experiments are not limited to screening libraries of compounds that are highly soluble in aqueous buffer. Typically, only the reference compound needs to be water soluble, and compounds with limited solubility can still be detected by their indirect effect on the signal of the reference compound. The reference molecule (that, for its role, is called the spy molecule) is generally water-soluble in order to avoid artefacts originating from possible non-specific interactions of the reference molecule with the receptor and with molecules of the mixtures to be screened. Titration NMR experiments with the reference molecule are typically first performed either at different ligand concentrations and fixed protein concentration or different protein concentrations and fixed ligand concentration (C. Dalvit et al., J. Am. Chem. Soc., 124, 7702-7709 (2002)). These experiments are used for the optimization of the screening setup conditions and for deriving the binding constant of the identified NMR hits from a single point measurement. Strong ligands are easily identified due to their large effect on the spy molecule. However, one limitation of the current ¹H NMR competition screening methods may be represented by the occurrence of spectral overlap between the reference and test compounds particularly when large chemical mixtures are screened. The use of NMR detection of other nuclei (e.g., ¹⁹F) reduces significantly these problems.

The present invention provides a variety of methods of identifying a ligand that interacts with a target molecule.

In certain embodiments, the method involves screening compounds to identify a ligand to a target molecule. The method includes: collecting a first ID ¹⁹F nuclear magnetic resonance spectrum of at least one test compound; exposing the at least one test compound to a target molecule; collecting a second 1D ¹⁹F nuclear magnetic resonance spectrum of the at least one test compound that has been exposed to the target molecule; and comparing the first and second spectra to determine a change in one or more of the resonances and identify at least one test compound that interacts with the target molecule.

In certain embodiments, the following steps are used: providing an ¹⁹F-labelled reference compound that interacts with the target molecule; collecting a ID ¹⁹F nuclear magnetic resonance spectrum of the ¹⁹F-labelled reference compound in the presence of the target molecule; providing at least one test sample (preferably a plurality of test samples), each test sample comprising at least one test compound; collecting a 1D ¹⁹F nuclear magnetic resonance spectrum of the ¹⁹F-labelled reference compound in the presence of each test sample and the target molecule; comparing the spectrum of the ¹⁹F-labelled reference compound in the presence of the target molecule to the spectrum of the ¹⁹F-labelled reference compound in the presence of each test sample and the target molecule to determine a change in one or more of the ¹⁹F-labelled reference compound resonances; and identifying at least one test compound that interacts with the target molecule, wherein the test compound displaces the ¹⁹F-labelled reference compound (typically, this results because the test compound has a binding affinity at least as tight as that of the reference compound).

Typically, a change in one or more of the ¹⁹F-labelled reference compound resonances involves an increase in signal intensity in at least one reference resonance. Preferably, a change in one or more of the ¹⁹F-labelled reference compound resonances involves a sharpening of at least one reference resonance.

If desired, before the acquisition of the ¹⁹F spectra, spin-echo type filters can be applied, as described in the Examples Section.

The optimum experimental conditions for any of the methods described herein can be determined as described in the Examples Section. Specifically, this typically involves the following steps being carried out prior to collecting a 1D ¹⁹F nuclear magnetic resonance spectrum of the ¹⁹F-labelled reference compound in the presence of the target molecule for use in the comparing step: collecting 1D ¹⁹F nuclear magnetic resonance spectra of the ¹⁹F-labelled reference compound in the presence of the target molecule at different concentrations of the target molecule or at different concentrations of the ¹⁹F-labelled reference compound. The information collected is used to determine the optimum experimental conditions for identifying at least one test compound that interacts with the target molecule.

A wide variety of pulse sequences can be used when collecting the 1D ¹⁹F NMR spectrum of the ¹⁹F-labelled reference compound in the presence of each test sample and the target molecule. For effective comparison of spectra, it is desirable to have the same experimental conditions; however, target compound and ¹⁹F-labelled reference molecule concentrations can be varied as long as the graphs with the titration experiments have been generated before the screening. Generally, the temperature and buffer conditions are the same, because a change in these experimental conditions can affect the binding constant of the reference compound.

In the generalized method described above for mixtures of two or more test compounds, identifying at least one test compound may preferably involve recording separate 1D ¹⁹F nuclear magnetic resonance spectra of the ¹⁹F-labelled reference compound in the presence of each test compound and the target molecule. This is followed by comparing the spectrum of the ¹⁹F-labelled reference compound in the presence of the target molecule to the spectrum of the ¹⁹F-labelled reference compound in the presence of each test compound and the target molecule to determine a change in the selected ¹⁹F-labelled reference compound resonance. The pulse sequences of these experiments are generally the same. Such experiments are typically referred to by those of skill in the art as deconvolution experiments.

The dissociation constant (i.e., binding affinity) of a test compound and/or a reference compound can be determined using NMR techniques if desired, although other well-known techniques can be used as well (e.g., isothermal titration calorimetry). Preferably, the reference compound binding affinity is evaluated using isothermal titration calorimetry or fluorescence spectroscopy, the specific details of which are well-known to one of skill in the art and are described in the Examples Section.

For example, in one NMR-based method, in addition to the above-listed steps in the generalized method, 1D ¹⁹F nuclear magnetic resonance spectra of the ¹⁹F-labelled reference compound in the presence of the target molecule at different concentrations of the ¹⁹F-labelled reference compound can be collected. Alternatively or additionally, 1D ¹⁹F nuclear magnetic resonance spectra of the ¹⁹F-labelled reference compound in the presence of the target molecule at different concentrations of the target molecule can be collected. This information can be used to determine the dissociation constant of the test compound as described in the examples.

For increasing the precision of any one method of the present invention, various techniques can be used. Typically, an internal control can be used, which can be a non-interacting compound.

An alternative to the use of a non-interacting molecule is the use of the ERETIC method (S. Akoka et al., Anal. Chem., 71, 2554-2557 (1999); and V. Silvestre et al., S. Anal. Chem., 73, 1862-1868 (2001). This technique relies on the electronic generation of a signal of a defined frequency, linewidth and amplitude. A pseudo-FID is acquired with the FID originating from the sample. The amplitude of this artificial signal is adjusted to become comparable to the intensity of the signal of the reference compound recorded in the absence of the protein. This amplitude value is then used for the titration and NMR-screening experiments and the signal intensity ratio of the real and artificial signal is measured. Adding an ERETIC signal is like adding a fake signal to normalize the signals.

For certain embodiments of the methods of the present invention, the reference compound is provided in combination with an ERETIC signal with defined linewidth, amplitude, and frequency. For these methods, collecting a 1D ¹⁹F nuclear magnetic resonance spectrum of the ¹⁹F-labelled reference compound in the presence of the target molecule includes collecting a spectrum of the ¹⁹F-labelled reference compound with the ERETIC signal in the presence of the target molecule; and collecting a 1D ¹⁹F nuclear magnetic resonance spectrum of the ¹⁹F-labelled reference compound in the presence of each test sample and the target molecule includes collecting a spectrum of the ¹⁹F-labelled reference compound with the ERETIC signal in the presence of each test sample and the target molecule.

For certain embodiments of the methods of the present invention, the ¹⁹F-labelled reference compound is provided in combination with an ¹⁹F-labelled non-interacting compound. For these methods, collecting a 1D ¹⁹F nuclear magnetic resonance spectrum of the ¹⁹F-labelled reference compound in the presence of the target molecule includes collecting a spectrum of the ¹⁹F-labelled reference compound and the ¹⁹F-labelled non-interacting compound in the presence of the target molecule; and collecting a 1D ¹⁹F nuclear magnetic resonance spectrum of the ¹⁹F-labelled reference compound in the presence of each test sample and the target molecule includes collecting a spectrum of the ¹⁹F-labelled reference compound and the ¹⁹F-labelled non-interacting compound in the presence of each test sample and the target molecule. Such non-interacting compounds act as controls in that they do not bind to the target molecule at the concentrations evaluated.

In combination with the competition binding experiments of the present invention, the WaterLOGSY method can be used to identify the reference compound, as well as other methods such as spectroscopic or biochemical assays, which are well known to one of skill in the art. Preferably, the reference compound can be identified by the following steps: collecting a WaterLOGSY nuclear magnetic resonance spectrum of a potential reference compound in the absence of the target molecule; collecting a WaterLOGSY nuclear magnetic resonance spectrum of the potential reference compound in the presence of the target molecule; and comparing the WaterLOGSY spectra to identify whether the potential reference compound interacts with the target molecule.

The WaterLOGSY method (also referred to as the Water-Ligand Observed via Gradient Spectroscopy Y) is based on the transfer of magnetization from the protons of bulk water to the protons of compounds that interact with target molecules (e.g., proteins). Using WaterLOGSY techniques, binding compounds are distinguished from nonbinders by the opposite sign of their water-ligand nuclear Overhauser effects (NOEs). The WaterLOGSY method is described in greater detail in International Publication No. WO 01/23330 (published Apr. 5, 2001), in C. Dalvit et al., J. Biomol. NMR, 18, 65-68 (2000), in Applicants' Representatives copending U.S. application Ser. No. 60/386,896, filed on Jun. 5, 2002 (Attorney Docket No. 01168.PRO1).

The target molecules that can be used in the methods of the present invention include a wide variety of molecules, particularly macromolecules, such as polypeptides (preferably, proteins), polynucleotides, organic polymers, and the like. These can be within a living cell or in a lysate.

“Polynucleotide” as used herein refers to a polymeric form of nucleotides of any length, either ribonucleotides or deoxynucleotides, and includes both double- and single-stranded DNA and RNA. A polynucleotide may include both coding and non-coding regions, and can be obtained directly from a natural source (e.g., a microbe), or can be prepared with the aid of recombinant, enzymatic, or chemical techniques. A polynucleotide can be linear or circular in topology. A polynucleotide can be, for example, a portion of a vector, such as an expression or cloning vector, or a fragment.

“Polypeptide” as used herein refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations, and the like.

The reference compound is one that interacts with the selected target molecule with a binding affinity sufficiently low. Relatively weakly interacting reference compounds are typically defined as those having a dissociation binding constant K_{D} of at least 10 micromolar.

The test compounds that can be evaluated can be any of a wide variety of compounds, which potentially have a wide variety of binding affinities to the target. Significantly, the method of the present invention has the ability to detect compounds that are relatively strong binders. The relatively strong binders are typically defines as those having a dissociation binding constant K_{D} of less than about 1 micromolar. Compounds that can be screened using the method of the present invention include, for example, plant extracts, fungi extracts, other natural products, and libraries of small organic molecules.

The present invention can screen for ligands from a library of compounds that have a broad range of solubilities (the methods are particularly amendable to compounds having very low solubilities). Significantly and advantageously, for certain embodiments, the present invention preferably involves carrying out a binding assay at relatively low concentrations of target (i.e., target molecule). Thus, preferred embodiments of the present invention allow for the detection of compounds that are only marginally soluble. Typically these compounds have a solubility in water of no greater than about 10 μM.

Preferably, the concentration of each test compound in each sample is no greater than about 100 μM, although higher concentrations can be used if desired. However, a significant advantage of the method of the present invention is that very low ligand concentrations (e.g., no greater than about 10 μM) can be used.

The exact concentrations and ratios of test compound to target molecule used can vary depending on the size of the target molecule, the amount of target molecule available, the desired binding affinity detection limit, and the desired speed of data collection. Preferably, the concentration of target molecule is about 100 nM to about 10 μM.

The solvents used for the test mixtures can be any of a wide variety as long as they do not degrade (e.g., denature) the target. Typically water and DMSO are used. Protonated solvents and detergents can be used.

If desired other components (e.g., buffers) can be added to the test mixtures for certain advantage, as is well known to one of skill in the art.

The present invention could also find useful applications for rapid screening of chemical mixtures (i.e., mixtures of two or more test compounds). Rapid screening techniques typically involve providing a plurality of test samples, each test sample comprising a mixture of two or more test compounds.

Once a ligand (preferably a high affinity ligand) has been identified and confirmed, its structure is used to identify available compounds with similar structures to be assayed for activity or affinity, or to direct the synthesis of structurally related compounds to be assayed for activity or affinity. These compounds are then either obtained from inventory or synthesized. Most often, they are then assayed for activity using enzyme assays. In the case of molecular targets that are not enzymes or that do not have an enzyme assay available, these compounds can be assayed for affinity using NMR techniques similar to those described above, or by other physical methods such as isothennal denaturation calorimetry. Compounds identified in this step with affinities for the molecular target of about 1.0×10⁻⁶ M or better are typically considered lead chemical templates.

In some instances, ligand binding is further studied using more complex NMR experiments or other physical methods such as calorimetry or X-ray crystallography.

Cryoprobe technology optimized for ¹H and ¹⁹F detection could further enhance the throughput of this screening process. In this case, the limiting factor will be the time required to change the sample, equilibrate the sample temperature, and shim the sample.

### EXAMPLES

Objects and advantages of this invention are further illustrated by the following examples, but the particular materials and amounts thereof recited in these examples, as well as other conditions and details, should not be construed to unduly limit this invention.

### Materials and Methods

For the first set of experiments in Example (I), the kinase domain (MW approximately 34000) of a Serine/Threonine p21-activated kinase was expressed as a GST fusion protein in E. Coli and purified to homogeneity after removal of the GST tag. NMR samples were in phosphate-buffered saline (PBS, code: P-3813, Lot 100K8211 from Sigma) pH 7.4. D₂O was added to the solution (8% final concentration) for the lock signal. The small molecules were prepared in concentrated stock solutions in deuterated DMSO and stored at 253 K.

For the second set of experiments in Example (II), fatty acid free human serum albumin (A-3782) was purchased from Sigma and used without further purification. NMR samples were in phosphate-buffered saline (PBS, code: P-3813, Lot 100K8211 from Sigma) pH 7.4 in the presence of 5 μM EDTA. D₂O was added to the solution (8% final concentration) for the lock signal. The small molecules were prepared in concentrated stock solutions in either deuterated DMSO or water and stored at 253 K.

### NMR

For the first set of experiments in Example (I), all NMR spectra were recorded at 293 K with a Varian Inova 600 MHz (564 MHz for ¹⁹F) NMR spectrometer equipped with a Sample Management System (SMS) autosampler. Water suppression in the ¹H detected experiments was achieved with the excitation sculpting sequence (T. -L. Hwang, J. Magn. Reson. A, 112, 275-279 (1995)). The two water selective 180° square pulses and the four pulsed field gradients of the scheme were 2.6 and 1 millisecond (ms) in duration, respectively.

For the second set of experiments in Example (II), all NMR spectra were recorded at 300 K with a Bruker Avance 600 NMR spectrometer operating at a ¹⁹F Larmor frequency of 564 MHz. A dual coil {¹⁹F}-{¹H} probe was used with the inner coil tuned to ¹⁹F and the outer coil tuned to ¹H frequency. The fluorine background of these probes does not interfere with the measurements. These signals are broad and therefore are not visible in the spectra of the reference and control molecule recorded with a spin-echo scheme. All the spectra were recorded with a weak Waltz-16 proton decoupling applied during the acquisition period. Typically 4-8 dummy scans were recorded for temperature equilibration. Carr-Purcell-Meibom-Gill schemes of different length and long 2τ interval were used before the acquisition period. Chemical shifts were referenced to trifluoroacetic acid.

### Fluorescence

Fluorescence measurements were acquired on a Jasco J-715 spectropolarimeter using an auxiliary photomultiplier tube positioned perpendicular to the excitation beam. The excitation wavelength was 310 nm (with a 5 mn bandwidth) and a 385 nm cut-off filter was employed. Affinity measurements were made using the same source of fatty acid free HSA as used for NMR experiments. Analyte and HSA solutions were prepared in phosphate-buffered saline (PBS, code: P-3813, Lot 100K8211 from Sigma) pH 7.4 in the presence of 5 μM EDTA. The buffer was filtered through a 0.2 μm filter prior to use. Albumin affinity was determined by aliquoting 2.0 mL of a 3 μM solution of analyte into a quartz cuvette, pathlength of 1.0 cm, and titrating the solution with HSA (stock concentration of 250 μM).

### ITC Experiments

Isothermal titration calorimetry experiments were performed using an OMEGA titrating microcalorimeter from Microcal, Inc. (Northampton, Mass.). The titrating microcalorimeter consisted of a sample and reference cell held in an adiabatic enclosure. The reference cell was filled with PBS. A 23 μM solution of HSA in PBS +2% DMSO was placed in the 1.37 mL sample cell. Analyte at 0.8 mM in the same buffer was held in a 250 μL syringe. Thirty injections (8 μL each and 12 seconds/injection) of analyte were made by a computer controlled stepper motor into the sample cell held at 25° C. The syringe stir rate was 400 rpm. Heat adsorbed or released with each injection was measured by a thermoelectric device connected to a Microcal nanovolt preamplifier. Titration isotherms for the binding interactions were comprised of the differential heat flow for each injection. Heat of dilution obtained by injecting analyte into PBS was negligible. Binding isotherms were fit to a single binding site model (T. Wiseman et al., Anal. Biochem., 179, 131-137 (1989)) using an iterative nonlinear least-squares algorithm included with the instrument.

### Example I

### Results and Discussion

### ¹⁹F Relaxation Theory

The longitudinal relaxation of ¹⁹F is not a good parameter for the competition binding experiments since it lacks the direct τ_{c} dependence necessary for identifying small molecules interacting with a macromolecule. However, the transverse relaxation rate R₂ represents an excellent parameter since it contains spectral densities calculated at 0 frequency (M. Goldman, Quantum Description of High-Resolution NMR in Liquids, Clarendon Press, Oxford, (1988)) for the heteronuclear ¹⁹F—¹H dipolar interactions and for the ¹⁹F chemical shift anisotropy (CSA) interaction as described by the equation:

$$\begin{matrix}{R}_{2}^{F} = \frac{{γ}_{F}^{2} {γ}_{H}^{2} {ℏ}^{2} {τ}_{c}}{20} {\sum }_{{H}_{i}}^{} \frac{1}{{r}_{FHi}^{6}} \{ 4 + \frac{1}{1 + {( {ω}_{F} - {ω}_{H} )}^{2} {τ}_{c}^{2}} + \frac{3}{1 + {ω}_{F}^{2} {τ}_{c}^{2}} + \\ \frac{6}{1 + {ω}_{H}^{2} {τ}_{c}^{2}} + \frac{6}{1 + {( {ω}_{F} + {ω}_{H} )}^{2} {τ}_{c}^{2}} \} + \\ \frac{2}{15} Δ {σ}^{2} {B}_{0}^{2} {γ}_{F}^{2} {τ}_{c} \{ \frac{2}{3} + \frac{1}{2 ( 1 + {ω}_{F}^{2} {τ}_{c}^{2} )} \}\end{matrix} \qquad ( 1 )$$

The Hᵢ correspond to all the protons of the reference compound and of the protein close in space to the fluorine atom, Δσ is the CSA of the ¹⁹F atom and B₀ is the strength of the magnetic field, γ_{H} and γ_{F} are the proton and fluorine gyromagnetic ratios, respectively, ω_{H} and ω_{F} are the proton and fluorine Larmor frequencies, respectively, τ_{c} is the correlation time and r_{FHi} is the internuclear distance between proton Hᵢ and the fluorine atom.

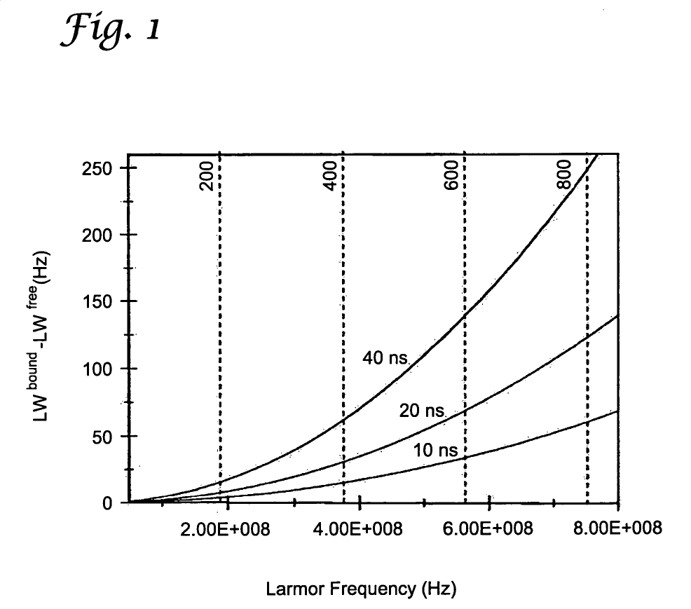

Owing to the large CSA of ¹⁹F (as much as few hundreds ppm) (J. T. Gerig, Methods in Enzymol., 177, 3-23 (1989); and J. T. Gerig, Prog. NMR Spectrosc., 26, 293-370 (1994)) it will contribute significantly to the transverse relaxation of the fraction of bound ligand. CSA contribution to relaxation is directly proportional to the square of the magnetic field. Therefore, the effect is more pronounced at higher magnetic fields. This can be appreciated in the simulation of FIG. 1 where the difference in ¹⁹F linewidth due to CSA contribution for a small molecule free in solution and when bound to a large macromolecule of different sizes is plotted as a function of the Larmor frequency. A Δσ of 100 ppm and a τ_{c} of 200 ps for the small molecule free in solution were used for the simulation. As can be appreciated from FIG. 1 the strongest magnetic fields available today are not optimal for the ¹⁹F experiments of strongly protein-bound ligands and of proteins selectively labeled with ¹⁹F due to the strong contribution of CSA. In contrast, the strong magnetic fields are particularly suited for the competition binding HTS experiments performed with a weak affinity reference molecule. The difference in ¹⁹F resonance linewidth of the reference molecule between the free and bound state increases with the strength of the magnetic field. In addition, the chemical shift difference for the fluorine ligand resonance between the free and bound state (δ_{free}-δ_{bound}) becomes larger. This results in a significant contribution of the exchange term R_{2, ex}

$${R}_{2 , ex} = \frac{[ EL ]}{[ {L}_{TOT} ]} {( 1 - \frac{[ EL ]}{[ {L}_{TOT} ]} )}^{2} \frac{4 {{π}^{2} ( {δ}_{free} - {δ}_{bound} )}^{2}}{{K}_{- 1}} \qquad ( 2 )$$

to the ¹⁹F linewidth of the reference compound (J. W. Peng,. J. Magn. Reson., 153, 32-47 (2001)). [EL]/[L_{TOT}] is the fraction of bound ligand and 1/K₋₁ is the residence time of the ligand bound to the protein.

### Selection of the Reference Compound and Screening Parameters

As the NMR screening experiments are typically carried out with a 10-100 fold excess of ligand over protein (resulting in only a small fraction of bound ligand), the terms in the preceeding section (Δσ, B₀, δ_{free}-δ_{bound}, 1/K₋₁, etc.) become important in the selection of the reference compound. The chemical shift of the ¹⁹F signal of the reference compound bound to the protein can be very different when compared to the chemical shift of the free ligand due to the contribution of the protein induced shift (J. T. Gerig, Prog NMR Spectrosc., 26, 293-370 (1994); and J. Feeney et al., J. Am. Chem. Soc., 118, 8700-8706 (1996)). Therefore even molecules having a medium binding affinity in the μM range could display a ¹⁹F spectrum with two distinct resonances, one for the bound form and the other for the free form. This arises from the fact that the difference in chemical shift of the two resonances is larger when compared to the exchange rate between the free and bound species. In the HTS only the signal at the frequency of the free ligand will be monitored since the concentration of the protein is to low (few hundreds nM to 1-5 μM) for allowing the observation of the very broad resonance of the bound form.

In order to assess the suitability of any given fluorinated ligand for competition screening and to determine the optimal experimental parameters, titration experiments for the candidate molecule are performed as a function of the fraction of bound ligand (C. Dalvit et al., J. Am. Chem. Soc., 124, 7702-7709 (2002)) by acquiring 1D ¹⁹F R₂ filtered experiments or simply 1D ¹⁹F experiments. For better sensitivity the experiments are recorded with ¹H decoupling during the acquisition period. Candidate compounds and experimental conditions are examined for their sensitivity and for the presence of a single fluorine resonance that exhibits significant perturbations upon binding to the target.

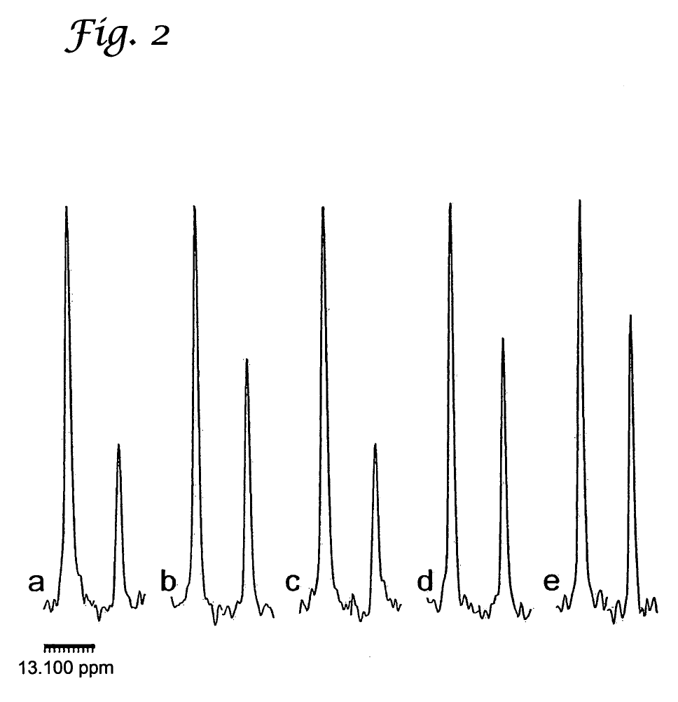

Once a suitable ligand has been identified and experimental conditions established, screening can then be carried out by monitoring changes in the transverse relaxation (either via the R₂ filtered experiments performed with CPMG or spin-echo schemes or simply by analysis of the linewidth) of the ¹⁹F signal of the reference molecule as shown in FIG. 2.

In this case, 50 μM of the moderate affinity ligand Compound A (K_{D}=10 μM) was used as the reference compound in the presence of 1.5 μM of PAK4. A significant increase in the signal of Compound A after the spin-echo scheme was observed in the presence of a compound mixture containing a high-affinity ligand (Compare FIG. 2a with 2b). This increase in signal indicates a decrease in binding of the reference molecule Compound A due to competition wiht a molecule contained in the mixture. Simple deconvolution experiments (2c and 2d) allows for the identification of Compound B as the active molecule.

When spin-echo type sequences are used, it is recommended to use a sufficiently long delay between the hard 180° pulses in order to take advantage of the exchange term of equation (2). This is possible since there are no homonuclear scalar couplings and the evolution under the heteronuclear scalar couplings is refocused. However, the delay should not be very long in order to avoid relaxation deriving from the inhomogeneity of the static magnetic field.

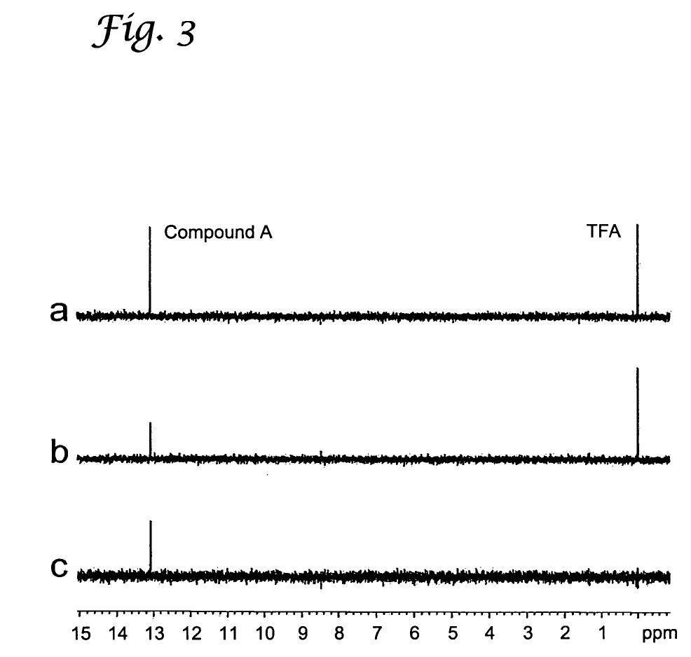

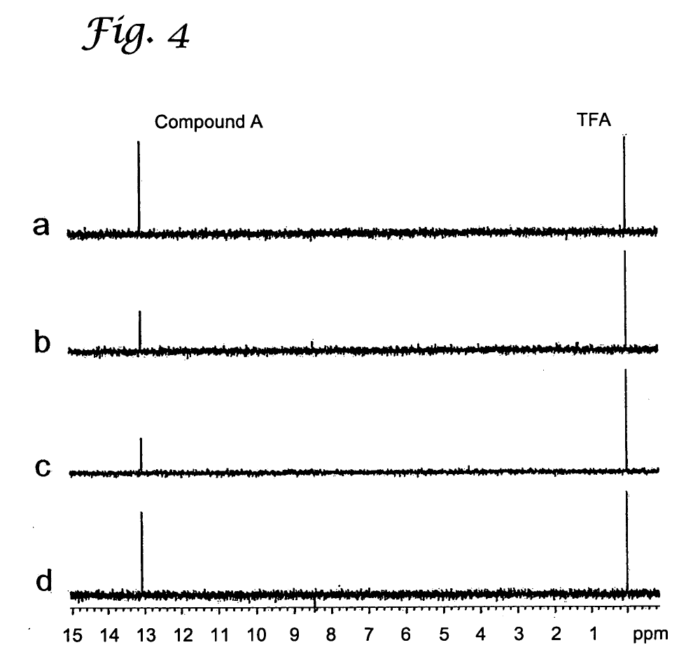

In addition to the fluorinated reference compound, it is advantageous to include a fluorinated small molecule that does not interact with the receptor (a control compound). FIG. 3 shows this principle where the ¹⁹F spectra of the spy molecule and of the non-interacting molecule are recorded in the absence and presence of the protein. While the signal of the spy molecule undergoes spectral changes, the signal of the small molecule will not change. This can be appreciated in the difference spectrum of FIG. 3. The signal of the non-interacting molecule represents an internal reference that can be used for calibrating with a single experiment the changes in the signal of the spy molecule. It should be pointed out that even if the small molecule had a weak interaction (mM range) with the receptor this would not interfere with the measurements. The concentration of the small molecule (10-30 μM) is orders of magnitude smaller when compared to the weak binding constant and therefore the fraction of compound bound to the receptor is negligible. In order to prove this trifluoroacetic acid (TFA) was chosen as the small molecule non-interacting with the receptor. It should be noted that some compounds in the screening may contain traces of TFA. Thus, while valid for the proof of concept presented here, an alternative control compound should be selected for more general application. The utility of using both a spy molecule and a control compound for lead identification through HTS and deconvolution is shown in FIG. 4. The six compound mixture does not affect the linewidth of the spy molecule resonance (FIG. 4c) and the signal of the reference compound Compound A is clearly less intense when compared to the signal of TFA. However, the presence of a strong competing molecule (Compound B) in the seven compound mixture results in a sharpening of the resonance of the spy molecule (FIG. 4d) and the two signals have now comparable intensity.

As described in C. Dalvit et al., J. Am. Chem. Soc., 124, 7702-7709 (2002), it is possible to derive the binding constant of the identified NMR-hit from the signal intensity ratio of the two ¹⁹F resonances plotted as a function of the fraction of bound ligand and the measurement of the signal intensity change of the reference molecule in the presence of a competing molecule. In this specific case the binding constant for the NMR-hit Compound B was determined to be 200±100 nM. When ¹⁹F experiments are used for the HTS it is important also to record the ¹H spectra in order to estimate the concentration of the compounds comprising the chemical mixtures and therefore derive a reliable value for the binding constant of the NMR hits.

A ¹H to ¹⁹F NOE step can also be applied in the ¹⁹F experiments before the acquisition period in order to transfer magnetization from the protons to the fluorine spin. This step can be performed in different ways. An enhancement of the ¹⁹F signal is observed for a small molecule not interacting with the large receptor. A very weak signal enhancement or a signal reduction, depending on the fraction of bound ligand, protein correlation time and on how the NOE step is performed is observed for a molecule interacting weakly with the receptor. These differences of the heteronuclear NOE can be used constructively in competition binding HTS experiments. When the spy molecule is displaced from the receptor in the presence of a competing molecule its ¹⁹F signal becomes more intense because of a smaller linewidth and signal enhancement via heteronuclear NOE.

### Example II

### Results and Discussion

### Theory

The sensitivity of ¹⁹F NMR signal is proportional to (γ_{F}/γ_{H})³ where γ_{F} and γ_{H} are the gyromagnetic ratio of fluorine and proton, respectively. Owing to the fact that ¹⁹F is the only stable fluorine isotope and has spin ½ its sensitivity is high, i.e. 0.83 times that of the proton. Fluorine signals appear as singlet resonances in the presence of proton decoupling and are therefore intense.

The ¹⁹F transverse relaxation represents an excellent parameter to be monitored for screening performed with competition binding experiments. A dipolar interaction between fluorine and a proton located at a certain distance is very similar in magnitude (0.88 times) to a dipolar interaction between two protons separated by the same distance. Therefore the dipolar contributions to the linewidth of a fluorine or proton signal of a reference molecule are similar. The transverse relaxation rate R₂ of the fluorine signal has an additional contribution originating from the large CSA interaction of the ¹⁹F atom and is given by the following equation (D. Canet, Nuclear Magnetic Resonance Concepts and Methods, John Wiley & Sons, Chichester, (1996)):

$${R}_{2}^{CSA} = \frac{2}{15} Δ {σ}^{2} ( 1 + \frac{{η}_{CSA}^{2}}{3} ) {B}_{0}^{2} {γ}_{F}^{2} {τ}_{c} \{ \frac{2}{3} + \frac{1}{2 ( 1 + {ω}_{F}^{2} {τ}_{c}^{2} )} \} \qquad ( 3 )$$

where Δσ is the CSA of the ¹⁹F atom and is given by Δσ=σ_{zz}−(σₓₓ+σ_{yy})/2. The different σ's are the components of the chemical shift tensor. The asymmetry parameter [symbol] _{CSA}=(3/2)(σₓₓ−σ_{yy})/Δσ and for an axially symmetric chemical shift tensor [symbol] _{CSA}=0. B₀ is the strength of the magnetic field, γ_{F} is the fluorine gyromagnetic ratio, ω_{F} is the fluorine Larmor frequency, and τ_{c} is the correlation time.

As discussed above in the first set of experiments (I), simulation performed assuming an axially symmetric CSA tensor and assuming an equal CSA for the free and bound state of a ligand, as shown in FIG. 1, indicates that the difference in linewidth of the ¹⁹F signal of the reference molecule between the free and bound state from just the CSA contribution alone can be very large (C. Dalvit et al., Comb. Chem. HTS, 5, 605-611 (2002) and Example (I) above). This difference increases with the size of the receptor and with the square of the magnetic field strength. High magnetic fields can lead to extremely broad linewidths (>200 Hz) for fluorine signals of either macromolecules (e.g., a protein selectively labeled with ¹⁹F) or strongly protein-bound ligands (W. E. Hull et al., J. Mol. Biol., 98, 121-153 (1975); J. T. Gerig, Methods in Enzymol., 177,3-23 (1989); and J. T. Gerig, Prog. NMR Spectrosc., 26, 293-370 (1994)). Such linewidths make the direct detection of fluorine resonances of the macromolecule or high-affinity ligands impractical for the purposes of screening. In contrast, the strong magnetic fields are particularly suited for competition binding experiments performed with a weak affinity reference molecule where the population averaging between the free and bound states results in an observed linewidth that can be manipulated and monitored (C. Dalvit et al., Comb. Chem. HTS, 5, 605-611 (2002) and Example (I) above).

The pulse sequences typically used employ a Carr-Purcell Meibom Gill (CPMG) spin-echo scheme (H. Y. Carr et al., Phys. Rev., 94, 630-638 (1954); and S. Meiboom et al., Rev. Sci. Instrum., 29, 688 (1958)) before the acquisition period. The signal intensity of the reference molecule at the end of the spin-echo scheme I₍ₙ₂_{_{τ}}₎ is given by the following equation (T. C. Farrar et al., Pulse and Fourier Transform NMR, Introduction to Theory and Methods, Academic Press, New York, 1971):

$${I}_{( n2 τ )} = {I}_{0} {ⅇ}^{- {γ}_{F}^{2} {G}^{2} {D}_{obs}^{( n2 τ )} \frac{{τ}^{2}}{3}} {ⅇ}^{- n2 τ {R}_{2 , obs}} \qquad ( 4 )$$

where I₀ is the signal intensity after the initial 90° pulse, 2τ is the interval between the train of 180° pulses, G is the inhomogeneity of the static magnetic field, γ_{F} is the gyromagnetic ratio of fluorine, and n is the number of cycles of the spin-echo scheme. D_{obs}, the observed translation diffusion coefficient for the weak affinity reference molecule, is given by the equation:

$${D}_{obs} = \frac{[ EL ]}{[ {L}_{TOT} ]} {D}_{bound} + ( 1 - \frac{[ EL ]}{[ {L}_{TOT} ]} ) {D}_{free} \qquad ( 5 )$$

where D_{bound} and D_{free} are the diffusion coefficients of the reference molecule in the bound and free states, respectively. [EL]/[L_{TOT}] and (1-[EL]/[L_{TOT}]) are the fraction of bound and free ligand, respectively.

R_{2,obs,} the transverse relaxation rate for the weak affinity reference molecule, is given by the equation:

$${R}_{2 , obs} = \frac{[ EL ]}{[ {L}_{TOT} ]} {R}_{2 , bound} + ( 1 - \frac{[ EL ]}{[ {L}_{TOT} ]} ) {R}_{2 , free} + \frac{[ EL ]}{[ {L}_{TOT} ]} {( 1 - \frac{[ EL ]}{[ {L}_{TOT} ]} )}^{2} \frac{4 {{π}^{2} ( {δ}_{free} - {δ}_{bound} )}^{2}}{{K}_{- 1}} \qquad ( 6 )$$

where R_{2,bound} and R_{2,free} are the transverse relaxation rate constants for the ligand in the bound and free states, respectively. The last term is the exchange term where δ_{bound} and δ_{free} are the isotropic chemical shifts of the fluorine resonance of the reference molecule in the bound and free states, respectively and 1/K₋₁ is the residence time of the ligand bound to the protein. Equation (6) is valid only when the experiments are performed with a long 2τ period (where τ>>1/K₋₁). Experiments recorded with τ<5/K₋₁ result in a reduced contribution of the exchange term to the observed transverse relaxation rate (Z. Luz et al., J. Chem. Phys., 39, 366-370 (1963); and A. Allerhand et al., H. S. J. Chem. Phys., 41, 2115-2126 (1964)).

Therefore screening is performed by using a long 2τ period. This is possible because the evolution under; the heteronuclear ¹H-¹⁹F scalar couplings is refocused at the end of the scheme. However, the 2τ period should not be very long in order to reduce signal attenuation originating from the spatial diffusion of the reference molecule (i.e., first exponential term of equation (4)).

### Selection of the Spy and Control Molecules

Table 1 reports the frequency of molecules containing a fluorine atom in three different commercially available chemical libraries. The table contains also the number of two substructures, monofluoro-benzene and trifluoromethyl-benzene, often used in these experiments. The large number of molecules containing a fluorine atom makes the selection of the spy and control molecules an easy task without recourse to chemical synthesis.

TABLE 1
Frequency of F containing molecules in different
commercially available libraries. ACD-SC (Available Chemical
Directory of Screening Compounds), MDDR (MDL Drug Data
Report), NCI (National Cancer Institute)

| Compound Collection | molecules with F | 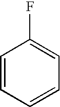 | 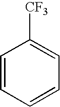 |
|---|---|---|---|
| ACD-SC | ˜12% | 153000 | 43000 |
| MDDR | ˜15% | 11000 | 3000 |
| NCI | ˜4% | 3000 | 1000 |

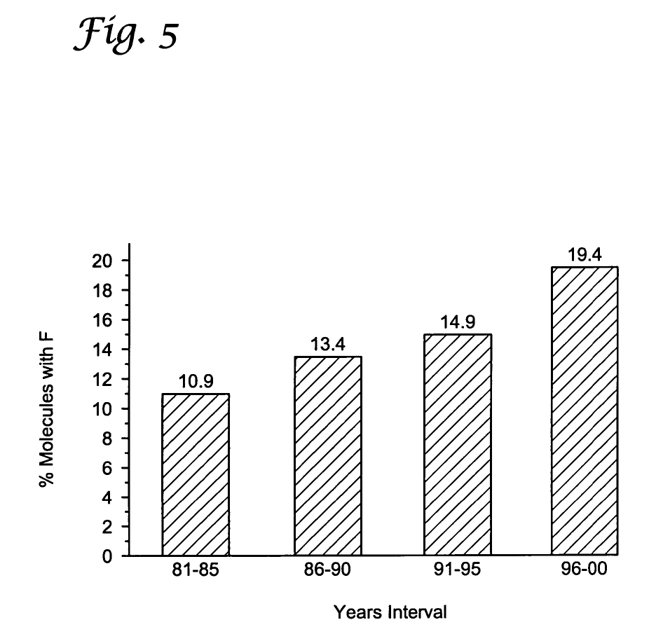

An interesting feature emerging from Table 1 is the large number of fluorine containing molecules present in the MDDR library. A chronological search within this library, as shown in FIG. 5, demonstrates that over the last years the percentage of compounds in development containing at least one fluorine atom has doubled. A steady increase from 10.9% in the 1981-1985 period to 19.4% in the 1996-2000 period is observed. The fluorine atom has been increasingly introduced in the process of lead optimization for improving potency, physical-chemical properties and metabolic stability against enzyme attack.

In the selection of the two molecules particular care should be paid to their solubility. The presence of a fluorine atom increases the lipophilicity of a compound. Molecules that are not very soluble in aqueous solution are not suitable for screening experiments since they might bind in a non-specific manner to the receptor. Therefore proton and fluorine spectra and proton WaterLOGSY spectra for the potential spy and control molecules are recorded in the absence of protein at a concentration typically 2 to 4 times higher (i.e., 100 to 200 μM) than the concentration used in the screening process. Only molecules that according to the NMR spectra are soluble and do not aggregate at these concentrations are considered as potential candidates for the reference and control molecules used for the screening.

### Molecules with a CF₃ Group

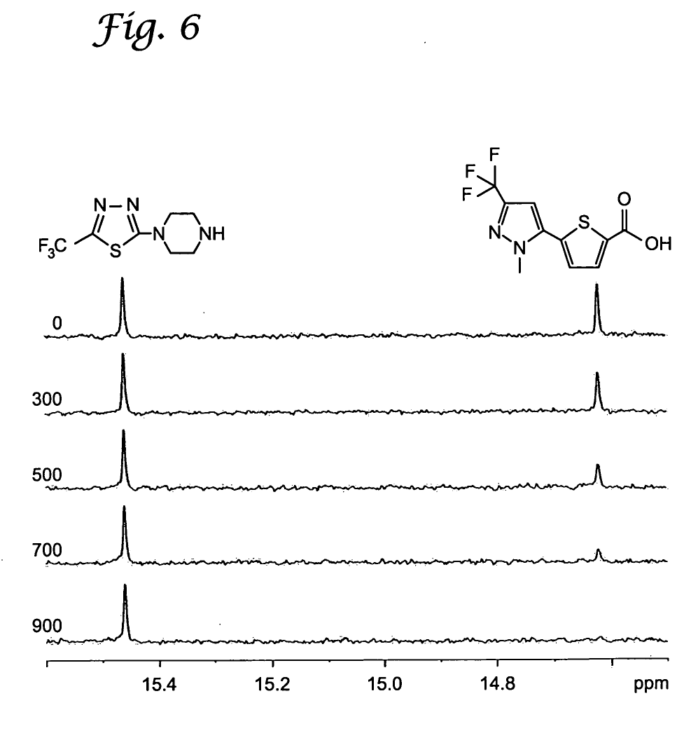

Reference molecules containing a CF₃ group have the advantage of high sensitivity of the fluorine signal. Typical spin-echo ¹⁹F spectra of the reference molecule 5-[1-methyl-3(trifluoromethyl)-1H-pyrazol-5-yl]-2-thiophenecarboxylic acid (1) and control molecule 1-[5-(trifluoromethyl)1,3,4-thiadiazol-2-yl]piperazine (2) recorded with proton decoupling during the acquisition period in the presence of different concentrations of HSA are shown in FIG. 6. ITC measurements performed with (2) did not find any evidence of binding to HSA (only heat of dilution was detected with 8 μL injections of 800 μM of (2) into 30 μM HSA) in agreement with the NMR results. A concentration of only 25 μM for both molecules was used for the NMR experiments. The low concentration of the reference molecule avoids problems arising from non-specific binding and aggregation. Disadvantages with these molecules are represented by the rapid rotation of the fluorine atoms about the C₃ axis of the group observed even in the bound state. This results in a limited difference in linewidth for the CF₃ signal of the reference molecule between the free and bound state.

### Molecules with a CF Group

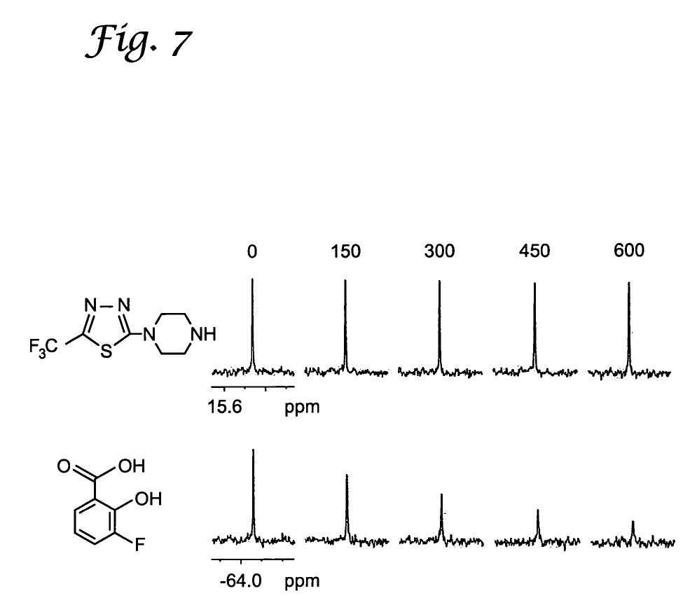

Molecules with a CF group are particularly suited for the competition ligand based screening experiments. The ¹⁹F CSA can be very large therefore increasing the difference in linewidth between the free and bound state of the reference molecule according to equation (3). For example the CSA for an aromatic CF ranges from 71 ppm for monofluoro-benzene to 158 ppm for hexafluoro-benzene (H. Raber et al., Chem. Phys., 26, 123-130 (1977)). In addition, the ¹⁹F CSA of the reference molecule in the bound state can increase due to an “ortho effect” or from the direct involvement of the fluorine atom in an hydrogen bond with the protein. These two phenomena also have the effect of rendering a large difference in the isotropical chemical shift for the free and bound state. For a weak affinity reference compound the exchange term of equation (6) can contribute significantly to the linewidth of the reference compound in the presence of the protein. The fluorine signal is usually scalar coupled with several protons and therefore for sensitivity improvement it is necessary to record the spectra with proton decoupling during acquisition. FIG. 7 shows typical spin-echo fluorine spectra for the reference molecule 2-hydroxy 3-fluorobenzoic acid (3) and control molecule (2) recorded with proton decoupling as a function of HSA concentration. A drawback with these molecules is the required higher concentration for the experiments. The spectra of FIG. 7 were recorded with a concentration for the reference molecule of 50 μM.

### Titration and Screening Experiments

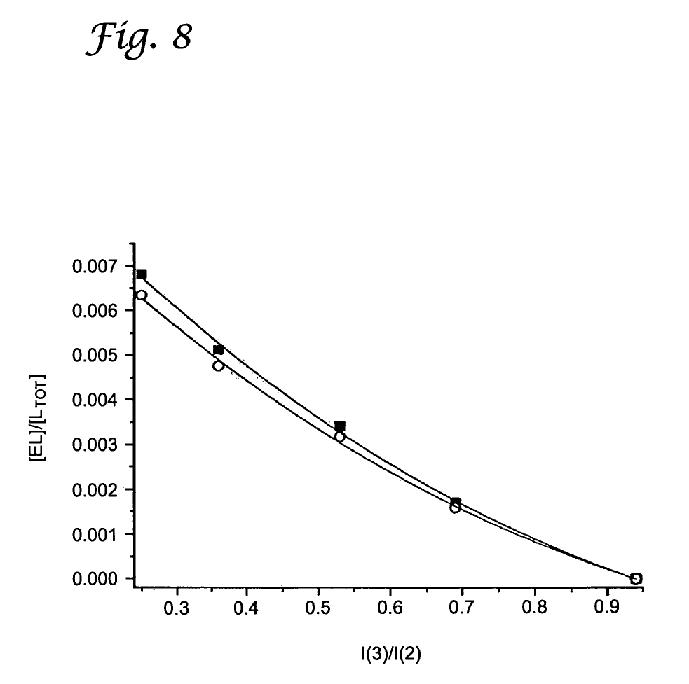

After the selection of the reference and control molecules, titration experiments as a function of the protein are recorded as shown in FIGS. 6 and 7. The intensity ratio of the two fluorine signals is plotted as a function of the fraction of protein-bound reference molecule as shown in the example of FIG. 8. The fraction of bound compound is calculated by using the dissociation binding constant derived from other techniques (e.g., ITC or fluorescence spectroscopy) as described in (C. Dalvit et al., J. Am. Chem. Soc., 124, 7702-7709 (2002); and C. Dalvit et al., Comb. Chem. HTS, 5, 645-650 (2002)). These techniques also provide the number of binding sites (n) for the reference molecule a parameter that is very important for the competition binding experiments. ITC measurements provided a n value that is close to 4 for (1) and close to 1 for (3). Therefore, although molecule (1) can still be used for screening purposes with some limitations in the interpretation of the experimental results, it cannot be used for deriving the binding constants of the NMR-hits. Molecule (3) represents a suitable reference molecule due to the presence of only one binding site on HSA at the concentration used in these experiments. According to its chemical structure, an aspirin analogue, its putative binding site would be the Sudlow site I (located in subdomain IIA) (T. Peters, Jr., All about Albumin Biochemistry, Genetics, and Medical Applications Academic Press, San Diego, U.S.A. 1996). Two different values for the fraction of protein bound reference molecule (3) are extracted in FIG. 8 using the two limit values of K_{D} determined by the experimental error. In this specific case, the ITC derived K_{D} for (3) was 41±3.3 μM and thus the two limit values of K_{D} correspond to 37.7 and 44.3 μM, respectively.

It should be pointed out that the NMR experiments could also be recorded in the presence of only the reference molecule (C. Dalvit et al., Comb. Chem. HTS, 5, 605-611 (2002) and above in Example (I)). In this case, however, two experiments have to be recorded: one without the CPMG sequence (i.e. 2nτ=0) and another with a CPMG with a long 2nτ. The signal intensity ratio extracted from these two spectra is then plotted as a function of the fraction of bound reference molecule.

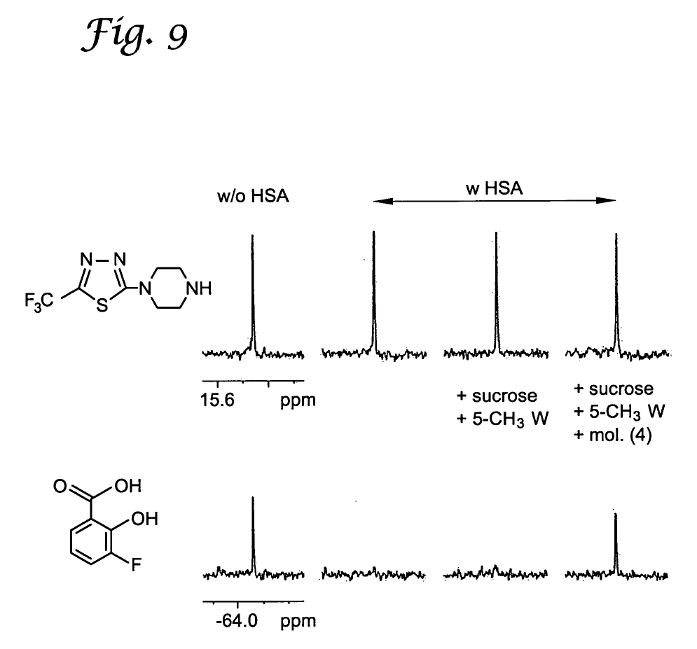

The graphs of FIG. 8 are then used for setting up the experimental conditions necessary for screening. FIG. 9 shows the screening process performed against HSA with (3) as reference molecule. For screening, a total spin-echo period (2nτ) was selected for which the signal of the reference molecule is approaching zero. The presence in the mixture of 5-CH₃ D,L Trp and sucrose (3^{rd} spectra from left), known as non-binders, do not alter the spectrum of the spy molecule. In contrast, the presence in the mixture of the warfarin derivative 4-hydroxy-3-[1-(p-iodophenyl)-3-oxobutyl] coumarin (4) (right) results in the reappearance of the signal of (3). This results, according to the graphs of FIG. 8, from the displacement of the reference compound from the protein. The extent of displacement can then be used to calculate the binding constant of the NMR-hit (C. Dalvit et al., J. Am. Chem. Soc., 124, 7702-7709 (2002); and C. Dalvit et al., Comb. Chem. HTS, 5, 645-650 (2002)) as described in Table 2.

TABLE 2
Single-Point NMR-derived binding constant for (4) and its comparison with measured
fluorescence value

| I (3)/I (2) | K_{D} | [I] | [L_{TOT}] | [E_{TOT}] | [EL]/[L_{TOT}] | [EL] | K_{D}^{app} | K_{I}^{NMR} | K_{I}^{fluo} |
|---|---|---|---|---|---|---|---|---|---|
| 0.698 | 44.3 | 25 | 50 | 0.6 | 0.00159 | 0.080 | 326.8 | 3.9 | 3.3 +/− 0.3 |
| 0.698 | 37.7 | 25 | 50 | 0.6 | 0.00171 | 0.086 | 300.4 | 3.6 | |

All the values for the concentration and binding constants are expressed in μM. K_{D} is the binding constant of (3) derived from ITC and K_{I}^{fluo} is the binding constant for (4) derived from fluorescence. [I], [L_{TOT}] and [E_{TOT}] are the concentration of (4), (3) and HSA, respectively. The K_{I}^{NMR} is the binding constant for (4) derived from the NMR measurements.

In order to derive a reliable value for the binding constant, one also has to record the proton spectrum. This is necessary for calculating the concentration of the NMR-hit by simply comparing the integral of a signal of the reference molecule for which the concentration is known with the integral of a signal of the NMR-hit. The NMR-derived K_{D} for (4) compares favorably with the value derived from a full titration fluorescence measurement. Since its first application (C. Dalvit et al., J. Am. Chem. Soc., 124, 7702-7709 (2002); and C. Dalvit et al., Comb. Chem. HTS, 5, 645-650 (2002)) binding constants have been calculated using this approach for several hundred compounds. For a pure competition binding mechanism and a single binding site, excellent agreement was observed between the single-point NMR derived binding constants and the full titration fluorescence- and ITC-derived binding constants. This NMR approach also allows the determination of high-affinity binding constants that would not be easily obtained with other NMR methods.

### Limit of Detection

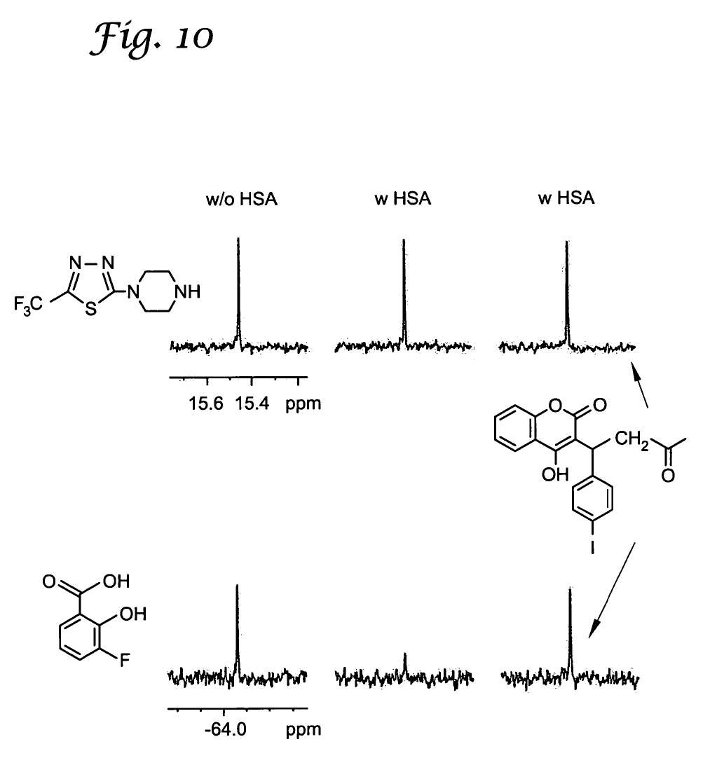

Owing to the large CSA and the large exchange contribution for a weak binding affinity reference molecule it is possible to significantly reduce the concentration of protein needed for screening. This can be appreciated in FIG. 10 where the screening is performed with (3) in the presence of HSA at a concentration of only 150 nM. Despite the large ratio [L_{TOT}]/[E_{TOT}] (=330) and the small ratio [EL]/[L_{TOT}] (=0.00165 using the K_{D} of 41 μM) it is possible to observe the effect of the small fraction of bound ligand and perform the screening at such low protein concentration. This probably represents a fortunate case. According to solid state NMR work, the presence of an OH group in the ortho position to the fluorine atom is responsible for a shift of 50 ppm in the component of the ¹⁹F chemical shift tensor perpendicular to the aromatic ring resulting in a large ¹⁹F CSA (H. Raber et al., Chem. Phys., 26, 123-130 (1977)). However, similar behavior was observed with other proteins and with reference molecules containing a para-fluoro benzyl moiety (data not shown). Therefore it is likely that the exchange term also contributes significantly to the observed transverse relaxation. The spectra reported in FIG. 10 were recorded with 128 scans and a total measuring time of 10 minutes. The use of cryoprobe technology optimized for ¹⁹F detection could further improve the detection limits. Protein concentrations as low as 50 to 100 nM could then be used. This will allow screening of a large number of chemical mixtures against proteins that cannot be expressed in high amount (e.g., membrane proteins). Fluorine spectra can be recorded very rapidly with cryoprobe technology. A conservative estimate of a two-fold sensitivity improvement with cryoprobe technology would translate into a four-fold reduction in acquisition time. Therefore the spectra of FIG. 10 could have been recorded in just 150 s, thus enhancing the throughput of this screening process. It should be pointed out that problems of radiation damping encountered in proton detected experiments recorded with cryoprobes are absent in the fluorine detected experiments because of the low concentration of the spy and control molecules.

### Screening in the Presence of Protonated Solvents and Detergents

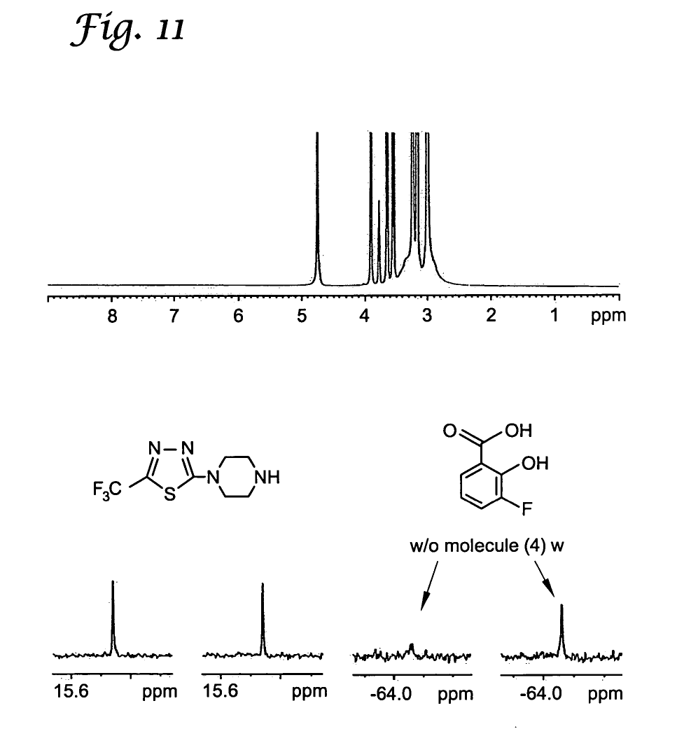

A particular advantage of the ¹⁹F ligand-based competition binding experiments is the possibility to perform the screening even in the presence of protonated solvents, buffers, or detergents. The proton spectrum of HSA in the presence of 100 mM HEPES and 1% glycerol is shown in FIG. 11. The intense signals of the buffer and glycerol mask the observation of the weak signals of the reference and control molecules necessary for performing the screening. These problems are not encountered in the ¹⁹F detection experiments. Therefore it is possible to perform the screening as shown in FIG. 11 even in these difficult experimental conditions. Because of these properties, fluorine ligand-based competition binding screening experiments are particularly advantageous to the screening of molecules against membrane proteins dissolved in SDS or other detergents. Once a suitable reference molecule has been identified, the ¹⁹F ligand-based competition binding NMR screening will provide reliable hits. The molecules that simply bind to the membranes and detergents and that appear as potential ligands in different assays will not be detected in the ¹⁹F experiments described here. Only molecules that compete with the reference molecule are identified. Finally, the experiments can also be used for screening of plant and fungi extracts, and for screening of molecules within living cells.

### Conclusion

Thus, the use of a weak affinity ligand containing a ¹⁹F atom in combination with the competition binding experiments permit rapid screening of large chemical mixtures against protein, DNA or RNA fragments. In addition, the method provides a direct determination of the binding constant of the identified NMR-hits.

With the ¹⁹F competition binding HTS experiments there are no problems of overlap and, because of the high sensitivity of ¹⁹F nucleus (0.83 times the ¹H sensitivity), it is possible to rapidly screen large chemical libraries or natural product extracts. In addition, the experiments can be performed for protein solutions in the presence of high concentrations of non-deuterated detergents therefore allowing HTS with membrane proteins. Furthermore, since resonances from the actual molecules screened are not utilized, only the spy and control molecules are required to contain a fluoro moiety. Thus, fluorine-based competition screening should find numerous uses in the pharmaceutical industry, and should further extend the impact of NMR-based screening on the drug discovery process.

The method is rapid and requires only a limited amount of protein and therefore compares favorably with the other established non-NMR techniques used in high-throughput screening. In addition, the method provides within a single experiment a meaningful value for the binding constant of the NMR-hit. The absence of overlap permits screening of large chemical mixtures originating from combinatorial chemistry, medicinal chemistry or natural product extraction. Screening against membrane proteins dissolved in different detergents is also possible with this approach. Finally, it is envisioned that these experiments can be extended to the screening of molecules against a receptor located within living cells.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein. Such examples and embodiments are presented by way of example only with the scope of the invention intended to be limited only by the claims set forth herein as follows.

What is claimed is:

1. A method of identifying a ligand to a target molecule, the method comprising:
providing an ¹⁹F-labelled reference compound that interacts with the target molecule;
collecting a 1D ¹⁹F nuclear magnetic resonance spectrum of the ¹⁹F-labelled reference compound in the presence of the target molecule;
providing a test sample comprising at least one test compound;
collecting a 1D ¹⁹F nuclear magnetic resonance spectrum of the ¹⁹F-labelled reference compound in the presence of each test sample and the target molecule;
comparing the spectrum of the ¹⁹F-labelled reference compound in the presence of the target molecule to the spectrum of the ¹⁹F-labelled reference compound in the presence of each test sample and the target molecule to determine a change in one or more of the ¹⁹F-labelled reference compound resonances; and
identifying at least one test compound that interacts with the target molecule, wherein the test compound displaces the ¹⁹F-labelled reference compound.

2. The method of claim 1 wherein the test compound has a binding affinity at least as tight as that of the reference compound.

3. The method of claim 1 wherein a change in one or more of the reference compound resonances comprises an increase in signal intensity in at least one reference resonance.

4. The method of claim 1 wherein identifying at least one test compound comprises recording separate 1D ¹⁹F nuclear magnetic resonance spectra of the ¹⁹F-labelled reference compound in the presence of each test compound and the target molecule.

5. The method of claim 1 further comprising:
collecting 1D ¹⁹F nuclear magnetic resonance spectra of the reference compound in the presence of the target molecule at different concentrations of the ¹⁹F-labelled reference compound; and
determining the dissociation constant of the test compound.

6. The method of claim 1 further comprising:
collecting 1D ¹⁹F nuclear magnetic resonance spectra of the ¹⁹F-labelled reference compound in the presence of the target molecule at different concentrations of the target molecule; and
determining the dissociation constant of the test compound.

7. The method of claim 1 wherein prior to collecting a 1D ¹⁹F nuclear magnetic resonance spectrum of the ¹⁹F-labelled reference compound in the presence of the target molecule for use in the comparing step, the method comprises:
collecting 1D ¹⁹F nuclear magnetic resonance spectra of the ¹⁹F-labelled reference compound in the presence of the target molecule at different concentrations of the target molecule or at different concentrations of the ¹⁹F-labelled reference compound; and
determining the optimum experimental conditions for identifying at least one test compound that interacts with the target molecule.

8. The method of claim 1 wherein the target molecule is a macromolecule.

9. The method of claim 8 wherein the macromolecule is a polypeptide or polynucleotide.

10. The method of claim 8 wherein the macromolecule is a protein.

11. The method of claim 1 wherein the reference compound binds to the target molecule with a binding affinity in the micromolar range.

12. The method of claim 11 wherein the binding affinity of the reference compound is determined by isothermal titration calorimetry or fluorescence spectroscopy.

13. The method of claim 1 further comprising a step of identifying the reference compound comprising:
collecting a WaterLOGSY nuclear magnetic resonance spectrum of a potential reference compound in the absence of the target molecule;
collecting a WaterLOGSY nuclear magnetic resonance spectrum of the potential reference compound in the presence of the target molecule; and
comparing the WaterLOGSY spectra to identify whether the potential reference compound interacts with the target molecule.

14. The method of claim 1 wherein the test sample comprises a mixture of two or more test compounds.

15. The method of claim 14 further comprising:
collecting a 1D ¹⁹F nuclear magnetic resonance spectrum of the ¹⁹F-labelled reference compound in the presence of each test compound and the target molecule; and
comparing the spectrum of the reference compound in the presence of the target molecule to the spectrum of the reference compound in the presence of each test compound and the target molecule to determine a change in the selected ¹⁹F-labelled reference compound resonance.

16. The method of claim 1 wherein the test compound has a binding affinity tighter than that of the reference compound.

17. The method of claim 1 wherein:
providing an ¹⁹F-labelled reference compound comprises providing an ¹⁹F-labelled reference compound and an ERETIC signal with defined linewidth, amplitude, and frequency;
collecting a 1D ¹⁹F nuclear magnetic resonance spectrum of the ¹⁹F-labelled reference compound in the presence of the target molecule comprises collecting a spectrum of the ¹⁹F-labelled reference compound with the ERETIC signal in the presence of the target molecule; and
collecting a 1D ¹⁹F nuclear magnetic resonance spectrum of the ¹⁹F-labelled reference compound in the presence of each test sample and the target molecule comprises collecting a spectrum of the ¹⁹F-labelled reference compound with the ERETIC signal in the presence of each test sample and the target molecule.

18. The method of claim 1 wherein:
providing an ¹⁹F-labelled reference compound comprises providing an ¹⁹F-labelled reference compound and an ¹⁹F-labelled non-interacting compound;
collecting a 1D ¹⁹F nuclear magnetic resonance spectrum of the ¹⁹F-labelled reference compound in the presence of the target molecule comprises collecting a spectrum of the ¹⁹F-labelled reference compound and the ¹⁹F-labelled non-interacting compound in the presence of the target molecule; and
collecting a 1D ¹⁹F nuclear magnetic resonance spectrum of the ¹⁹F-labelled reference compound in the presence of each test sample and the target molecule comprises collecting a spectrum of the ¹⁹F-labelled reference compound and the ¹⁹F-labelled non-interacting compound in the presence of each test sample and the target molecule.

19. A method of screening compounds to identify a ligand to a target molecule, the method comprising:
collecting a first 1D ¹⁹F nuclear magnetic resonance spectrum of at least one test compound;
exposing the at least one test compound to a target molecule;
collecting a second 1D ¹⁹F nuclear magnetic resonance spectrum of the at least one test compound that has been exposed to the target molecule; and
comparing the first and second spectra to determine a change in one or more of the resonances and identify at least one test compound that interacts with the target molecule.

20. The method of claim 1 wherein providing a test sample comprises providing a plurality of test samples, each test sample comprising at least one test compound.
